(19) United States
(12) Patent Application Publication
(10) Pub. No.: US 2008/0109035 A1
(43) Pub. Date: May 8, 2008

(54) Methods and Compositions for Repairing Common Peroneal Nerve Lesions

(76) Inventor: Henrich Cheng, Taipei (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(21) Appl. No.: 11/930,961

(22) Filed: Oct. 31, 2007

Related U.S. Application Data
(60) Provisional application No. 60/863,651, filed on Oct. 31, 2006.

(51) Int. Cl. A61K 33/06; A61K 33/10; A61P 25/00

(52) U.S. Cl. 606/214; 424/682; 424/687

(57) ABSTRACT

Methods and compositions are provided for repairing common peroneal nerve (CPN) lesions and enhancing functional recovery of a damaged CPN. The methods of the present invention include applying a fibrin glue mixture to the area of a surgically repaired CPN. The fibrin glue mixture contains growth factor, fibrinogen, aprotinin and divalent calcium ions.

### CROSS-REFERENCE TO RELATED APPLICATIONS

This application claims the benefit of Provisional Application No. 60/863,651, filed Oct. 31, 2006, the disclosure of which is incorporated herein by reference.

### BACKGROUND OF THE INVENTION

The present invention relates to methods and kits for repairing common peroneal nerve (CPN) lesions and enhancing functional recovery of a damaged CPN.

Neurological damage may limit functional outcome. For this reason, injuries to the nervous system require careful management to maximize recovery. The degree to which a nerve is damaged imposes substantial influence on its present function and potential for recovery. After complete axonal transection, the neuron undergoes a series of degeneration processes, followed by attempts at regeneration. The time-dependent decline of the ability of motoneurons to regenerate the axons after axotomy is one of the principal affecting factors to poor recovery after peripheral nerve injury, and the decline in neurotrophic support may be partially responsible for this effect.

The first growth factor, nerve growth factor (NGF), discovered in the 1950s, promotes the survival and differentiation of sympathetic and sensory neurons. Many subsequent attempts were made to induce nerve regeneration at nerve defects using various neurotrophic factors: Glial-derived neurotrophic factor (GDNF) has a trophic effect on dorsal root ganglion cells as well as on motoneurons and autonomic neurons. Ciliary neurotrophic factor (CNTF) promotes survival of motoneurons in vitro and in neonatal animals following axotomy. Acidic fibroblast growth factor (aFGF) treatment prevents motoneuron loss, improves corticospinal tract regeneration, and contributes to angiogenesis in vitro. In our previous studies, hindlimb function was restored after surgical repair of transected spinal cord with aFGF in rat model (Cheng et al., 1996, Science 273: 510-513). There was also functional regeneration after repairing the transected cervical roots with nerve graft and aFGF in adult rats (Chuang et al., 2002, Life Sci 71: 487-496; Huang et al., 2003, Exp Neurol 180: 101-109; Lee et al., 2004, Life Sci 74: 1937-1943). Two case reports also demonstrated functional recovery in patients with chronic paraplegia due to spinal cord injury or transverse myelitis after treatment with aFGF (Cheng et al., 2004, Spine 29: E284-288; Lin et al., 2006, Spinal Cord 44: 254-257).

Common peroneal nerve (CPN) lesion is the most common mononeuropathy of lower limbs. Because of high rates of spontaneous resolution and poor surgical outcome, numerous investigators advocated non-surgical treatment. Recent work by Garozzo et al. suggested that spontaneous recovery may occur when the nerve is in continuity without severe damage to its connective tissue elements (Garozzo et al., 2004, J Neurosurg Sci 48: 105-112). However, where there is severe damage, surgical repair is mandatory.

U.S. patent application publication 2004/0267289 A1 of applicant discloses a method of connecting a portion of the peripheral nervous system to a portion of the central or peripheral nervous system of a vertebrate using a fibrin glue mixture of growth factor, fibrinogen, aprotinin, and divalent calcium ions.

Up to now, there have been no clinical trials concerning the effect of aFGF on human peripheral nerve lesions. In addition, there remains a need for a means to effectively repair peripheral nerve lesions and, furthermore, to enhance the functional recovery of damaged peripheral nerves.

### BRIEF SUMMARY OF THE INVENTION

The present invention is based on the discovery that common peroneal nerve (CPN) lesions can be more effectively repaired by using a fibrin glue mixture after a surgical repair to restore the function of the damaged CPN.

Accordingly, an embodiment of the invention features a method for repairing a CPN lesion comprising: i) surgically repairing the CPN at or near the CPN lesion; and ii) applying an effective amount of a fibrin glue mixture to the surgically repaired area of the CPN, wherein the fibrin glue mixture comprises growth factor, fibrinogen, aprotinin and divalent calcium ions.

Another embodiment of the invention features a method for enhancing the functional recovery of a surgically repaired CPN comprising applying an effective amount of fibrin glue mixture to the surgically repaired area of the CPN, wherein the fibrin glue mixture comprises growth factor, fibrinogen, aprotinin and divalent calcium ions.

A further embodiment of the invention comprises a kit comprising a fibrin glue mixture that comprises growth factor, fibrinogen, aprotinin and divalent calcium ions, and instructions for using the fibrin glue mixture in a surgical repair of a CPN lesion.

According to the present invention, the surgical repair may involve at least one of axotomy, nerve graft, and neurolysis.

In a preferred embodiment of the present invention, the fibrin glue mixture comprises acidic fibroblast growth factor (aFGF), fibrinogen, aprotinin and divalent calcium ions.

### BRIEF DESCRIPTION OF THE SEVERAL VIEWS OF THE DRAWINGS

The foregoing summary, as well as the following detailed description of the invention, will be better understood when read in conjunction with the appended drawing. For the purpose of illustrating the invention, there is shown in the drawing embodiments which are presently preferred. It should be understood, however, that the invention is not limited to the precise arrangements and instrumentalities shown.

In the drawing:

FIG. 1 is a series of bar graphs showing the mean and standard deviations of average muscle strength of each of the three groups at baseline, 1st and 2nd follow-up evaluations, with group 1 having more effective repair of the CPN lesion according to one embodiment of the invention.

### DETAILED DESCRIPTION OF THE INVENTION

Various publications, articles and patents are cited or described in the Background of The Invention and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. In this invention, certain terms are used frequently, which shall have the meanings set forth as follows. These terms may also be explained in greater detail later in the specification.

It must be noted that as used herein and in the appended claims, the singular forms “a,” “an,” and “the” include plural reference unless the context clearly dictates otherwise. Thus, for example, a reference to “a fibrin glue mixture” is a reference to one or more fibrin glue mixtures and includes equivalents thereof known to those skilled in the art and so forth.

As used herein, the term “common peroneal nerve” or “CPN” means the smaller of the branches into which the sciatic nerve divides passing obliquely outward and downward from the popliteal space and to the neck of the fibula where it divides into the deep peroneal nerve and the superficial peroneal nerve that supply certain muscles and skin areas of the leg and foot, called also lateral popliteal nerve, peroneal nerve. The common peroneal nerve, including its deep branch, is the most commonly injured nerve. A CPN lesion or a lesion to the CPN may be due to entrapment, compression, stretch injury, ischemia, infection, or inflammation of the CPN. For example, being located in a lateral subcutaneous position at the fibular neck; a lesion to CPN causes a loss of ability to dorsiflex the foot (“foot drop”).

As used herein, the term “growth factor” includes a substance that promotes growth and development by directing cell maturation and differentiation and by mediating maintenance and repair of tissues. In particular embodiments, the growth factor can be any of a complex family of polypeptide biological factors. Each of the growth factors herein can be a natural substance produced by the body of an animal, including but not limited to, human, rat, mouse, pig, dog and monkey, or obtained from food, such as vitamins and minerals. It can also be a recombinant product produced by a recombinant host, such as a recombinant bacterium, from the growth factor gene.

A person skilled in the art will understand that each of the growth factors herein also includes a structural and/or functional derivative of the naturally occurring growth factor, such as a fragment of the growth factor or a chemically modified growth factor, that maintains the biological activity of the growth factor. The growth factor can be chemically modified to achieve certain desirable properties, such as enhanced stability or bioavailability. Common modifications to a protein include, for example, glycosylation, lipid attachment, sulfation, gamma-carboxylation of glutamic acid residues, hydroxylation and ADP-ribosylation.

As used herein, the term “fibrinogen” means a protein that is converted into fibrin by the action of thrombin especially during blood clot formation. The fibrinogen can be a natural substance produced in the liver of an animal, including but not limited to, human, rat, mouse, pig, dog and monkey. It can also be a recombinant product produced by a recombinant host, such as a recombinant bacterium, for the fibrinogen gene. The fibrinogen further includes any structural and/or functional derivative of the naturally occurring fibrinogen, such as a fragment of or a chemically modified fibrinogen, that maintains the biological activity of the fibrinogen.

As used herein, the term “aprotinin” refers to a polypeptide that is known for its protease-inhibiting properties, especially in inhibiting several serine proteases, such as trypsin, chymotrypsin, kallikrein, and pepsin. The aprotinin can be a natural substance produced in the organ of an animal, including but not limited to, human, rat, mouse, pig, dog and monkey. It can also be a recombinant product produced by a recombinant host, such as a recombinant bacterium, for the aprotinin gene. The aprotinin further includes any structural and/or functional derivative of the naturally occurring aprotinin, such as a fragment of or a chemically modified aprotinin that maintains the biological activity of the aprotinin.

The term “effective amount” as used herein, means that amount of a fibrin glue mixture that when applied to the surgically repaired area of a damaged CPN, more effectively repairs the damaged CPN or enhances the functional recovery of the damaged CPN as compared to the surgical repair of the damaged CPN alone.

In one embodiment, the invention relates to a method of repairing a CPN lesion comprising the steps of i) surgically repairing the CPN at or near the CPN lesion; and ii) applying an effective amount of a fibrin glue mixture to the surgically repaired area of the CPN, wherein the fibrin glue mixture comprises growth factor, fibrinogen, aprotinin and divalent calcium ions.

Procedures for surgical repair of a CPN lesion are known to a killed artisan. One skilled in the art will recognize that alternative procedures, such as that of neurolysis, axotomy, nerve graft, or the combinations thereof, can be performed to surgically repair a CPN lesion. An exemplary surgical repair procedure can include, for example, the steps of: administering anesthesia to the patient; performing incision at the CPN lesion; dissecting and looping the CPN and other nerves; and identifying and resecting the fibrotic bands. One skilled in the art will recognize that some or all of these steps can be substituted with alternative steps that are broadly equivalent.

In another embodiment, the invention relates to a method of enhancing the functional recovery of a surgically repaired CPN comprising the step of applying an effective amount of a fibrin glue mixture to the surgically repaired area of the CPN, wherein the fibrin glue mixture comprises growth factor, fibrinogen, aprotinin and divalent calcium ions.

One skilled in the art will recognize that the effective amount of the fibrin glue mixture can vary with multiple factors, such as the subject, the type of CPN lesion, the surgical procedure used to repair the CPN lesion, and the concentration of each of the components of the fibrin glue mixture, etc. Standard procedures, such as those described in the Examples below, can be performed to evaluate the effect of the fibrin glue mixture to a surgically repaired CPN, thus allowing a skilled artisan to determine the effective amount. In particular embodiments, the effective amount of the fibrin glue mixture covers the entire surgically repaired area of the CPN. In other embodiments, the effective amount of the fibrin glue mixture covers a portion of the surgically repaired area of the CPN.

The surgically repaired area of the CPN includes the surgically repaired CPN and its surrounding area. In particular embodiment, the surgically repaired area of the CPN includes the perineurium, which is the connective tissue sheath that surrounds a bundle of nerve fibers.

According to the invention, the fibrin glue mixture can be applied to the surgically repaired area by means known to a skilled artisan. The components of the fibrin glue mixture can be applied to the surgically repaired area of the CPN simultaneously or separately. According to one example of the invention, the fibrin glue mixture comprises a growth factor, a fibrinogen, an aprotinin and divalent calcium ions. In a particular embodiment, the fibrin glue is prepared before use by mixing the fibrinogen and aprotinin in ration of 1:1 to transform into pellucid, colloidal liquid. Finally, it is added divalent calcium ions to gain the coagulated solid product. The fibrin glue mixture is subsequently applied to the surgically repaired area of the CPN to form a glue cast.

In particular embodiments of the invention, the growth factor contained in the fibrin glue mixture includes, but is not limited to, a glial cell line-derived neurotrophic factor (GDNF), a transforming growth factor-beta, a fibroblast growth factor (FGF), a platelet-derived growth factor, an epidermal growth factor, a vascular endothelial growth factor (VEGF), a neurotrophin, or a combination of any two or more of the growth factors listed herein. In particular embodiments of the invention, the neurotrophin is selected from a nerve growth factor (NGF), a brain-derived neurotrophic factor (BDNF), a neurotrophin 3 (NT 3), a neurotrophin 4 (NT 5), a neurotrophin 5 (NT 4), and a combination of any two or more of the neurotrophins listed herein. In one example of the invention, the growth factor comprises a member of the FGF family, including but not limited to, an acidic fibroblast growth factor (aFGF) and a basic fibroblast growth factor (bFGF). The FGF family members bind heparin and have been implicated in diverse biological processes, such as limb and nervous system development, wound healing, and tumor growth. Most preferably, the growth factor comprises an aFGF, also named FGF1 or FGF alpha, which acts as a mitogen for a variety of mesoderm- and neuroectoderm-derived cell in vitro, thus is thought to be involved in organogenesis.

The concentration of growth factor in the fibrin glue mixture is from about 0.0001 to about 1000 milligram (mg) per milliliter (ml) of the total volume of the fibrin glue mixture (mg/ml). According to one example of the invention, the mixture comprises about 1 mg/ml of aFGF in the fibrin glue mixture.

The concentration of fibrinogen in the fibrin glue mixture is preferably about 10 to about 1000 mg per milliliter of the total volume of the fibrin glue mixture (mg/ml), such as about 100 mg/ml.

The concentration of aprotinin in the fibrin glue mixture is preferably about 10 to about 10000 Kilo International Unit (KIU) per milliliter of the total volume of the fibrin glue mixture (KIU/ml), such as about 200 KIU/ml.

According to the present invention, the divalent calcium ions presented in the fibrin glue mixture can be any physiologically acceptable calcium compound that dissociates in water and releases divalent calcium ions, such as calcium chloride and calcium carbonate. The concentration of calcium chloride in the fibrin glue mixture is preferably about 1 to about 100 micromole (μmol) per milliliter of the total volume of the fibrin glue mixture (mM), such as about 8 mM.

In particular embodiments, the fibrin glue mixture used in the present invention comprises aFGF, fibrinogen, aprotinin and calcium chloride. Preferably the fibrin glue mixture used in the present invention comprises about 1 mg/ml aFGF, about 100 mg/ml fibrinogen, about 200 KIU/ml aprotinin, and about 8 mM calcium chloride.

In other embodiments, the fibrin glue mixture used in the present invention further comprises one or more additional substances for enhancing surgical repair of CPN lesion, which is selected from, but is not limited to, the group consisting of a steroid, e.g. methylprednisone; a cytokine; a chemokine; a proteinase, e.g. a metalloproteinase; an extracellular matrix molecule, e.g. laminin or tenascin; a guidance molecule, i.e. a molecule that attracts or repels the migration of a cell, e.g. netrin, semaphorin, neural cell adhesion molecule, cadherin, thioredoxin peroxidase or Eph ligand; an anti-angiogenic factor, e.g. angiostatin, endostatin, TNP-470 or kringle 5; a neuroprotective agent, e.g. N-methyl D-aspartate (NMDA), a non-NMDA antagonist, a calcium channel blocker, nitric oxide synthase (NOS), a NOS inhibitor, peroxynitrite scavenger or a sodium channel blocker; and a Nogo gene polypeptide and antibodies that specifically bind to the polypeptide.

The fibrin glue mixture used in the method of the present invention can also optionally include a cell or cell suspension for facilitating repair, such as Schwann cells, bone marrow cells, blood cells, stem cells or olfactory ensheathing glial (OEG) cells.

Another general aspect of the invention is a kit comprising a fibrin glue mixture that comprises growth factor, fibrinogen, aprotinin and divalent calcium ions, and instructions for using the fibrin glue mixture in a surgical repair of a CPN lesion. Such kit can be used for more effective surgical repair of a CPN lesion or to enhance the functional recovery of a surgically repaired CPN. Such a kit preferably comprises a compartmentalized carrier suitable to hold in close confinement at least one container containing the fibrin glue mixture. In preferred embodiments, the kit comprising a fibrin glue mixture that comprises aFGF, fibrinogen, aprotinin and calcium chloride, and an instruction for using the fibrin glue mixture in a surgery repair of a CPN lesion.

The present invention is further illustrated by the following examples, which are provided for the purpose of demonstration rather than limitation.

### EXAMPLE

### Functional Recovery of Patients with CPN Lesions Participants

Patients with drop foot were recruited prospectively from the Department of Neurosurgery, Taipei Veterans General Hospital between July 2001 and June 2004. All patients were referred to the electrophysiologic laboratory of the Department of Physical Medicine and Rehabilitation for needle electromyography and nerve conduction studies of lower extremity. The inclusion criteria of the study were an initial electrophysiological diagnosis of CPN lesions with axonal loss. The exclusion criteria were: neuropraxic lesions by electrophysiological study, sustained two-level lesions such as L5 radiculopathy or sciatic nerve injury, comprised with any diabetic or metabolic disease or any other neuromuscular disorders in the affected limb which may complicate with peripheral nerve lesions. These participants received surgical repair with fibrin glue comprising aFGF. We collected the data of motor and sensory function, as well as electrophysiological examination before the surgical intervention, 6 months after the intervention (1st follow up) and 12 months after the intervention (2nd follow up). This study adhered to the Occupational Health and Safety Administration regulations, and was approved by the local institutional board and the National Organization for Human Research. Informed consents were obtained from all patients.

Data of the control groups were collected through a retrospective medical chart review of patients diagnosed to have CPN lesions with axon loss at the electrophysiological laboratory of Department of Physical Medicine and Rehabilitation between January 1990 to June 2000. Patients with incomplete chart records on motor and sensory functions or those without follow-up electrophysiological examinations were excluded. A total of 24 patients met the same inclusion and exclusion criteria as aforementioned. Among them, 8 patients received surgical repair and constituted group 2. The remaining 16 patients, who did not receive any surgical intervention, constituted group 3. Since the data was collected retrospectively in groups 2 and 3, the follow-up time may vary. Therefore, we collected the data of follow-up evaluation done between 5 to 7 months after the intervention as 1st follow up evaluation and data obtained from the evaluations done 12 months or more postoperatively as 2nd follow up evaluation.

### Evaluation Methods

Motor Function

Muscle strength was scored, according to the manual muscle testing criteria of the Medical Research Council, using a 6-point scale (range 0-5). Grade 0 represented no muscle activity, and grade 5 represented normal muscle strength. Muscle strength of the anterior tibialis, peroneus longus and extensor hallucis longus were measured as ankle dorsiflexion, ankle eversion and big toe dorsiflexion. The extent of initial injury of the nerve and the consequent recovery of each muscle may vary in the same patient; therefore, the muscle strength scores of anterior tibialis, extensor hallucis longus and peroneus longus were averaged to represent the overall motor function of the nerve.

Sensory Function

Light touch and pinprick sensation over mid- and lower lateral calf were evaluated to determine the sensory function of superficial peroneal nerve: “0” represents absent sensation, “1” represents impaired sensation, and “2” represents normal sensation.

Electrophysiological Studies

A comprehensive electrophysiological examination including nerve conduction study and needle electromyography of the affected limb was performed using a disposable Dantec DCN 27 concentric needle. The electrophysiologic diagnosis was based on abnormalities detected on needle examination in muscles innervated by CPN, such as the anterior tibialis, peroneus longus, or extensor hallucis longus. These abnormalities include increased insertion activity, increased spontaneous activity, neurophatic motor units, or decreased recruitment of motor units. The electrophysiological study was considered abnormal if increased insertion activity or spontaneous activity, abnormal motor units, or recruitment patterns was observed on needle examination. Nerve injuries were labeled as “loss of axon continuity” if no motor units were observed during active voluntary recruitment in the presence of spontaneous activity. The extent and severity of the damage were graded according to a modified version of Dumitru's (Dumitru, 1995, Electrodiagnostic Medicine. Hanley & Belfus, Inc.) and Wilbourn's (Wilbourn, 1985, Neurol Clin 3: 511-529) scale as follows:

Mild (score=1): normal sensory nerve action potential (SNAP) amplitude and normal compound muscle action potential (CMAP) amplitudes of nerve conduction studies, with occasional denervation and normal motor unit recruitment on electromyography.

Moderate (score=2): slight to profound decrease in SNAP amplitude and normal to slight decrease in CMAP amplitude, as compared with the amplitudes of evoked action potentials of the normal side, with constant denervation and normal to slight decrease in motor unit recruitment.

Severe (score=3): absent SNAP and profound decrease to absent CMAP amplitude, with marked denervation and discrete to no motor unit recruited.

Surgical Procedures

In group 1 patients, surgical repair with fibrin glue and aFGF was performed. Under general anesthesia, the patient was put on prone position. A 15 cm curvilinear incision was done from the middle of popliteal fold extending down to lateral aspect near fibular head. After the wound was deepened, the common peroneal nerve and tibial nerve were dissected and looped. The fibrotic bands were identified and then carefully resected. The soft tissue around the nerve was dissected to avoid compression. After checking bleeders, the wound was irrigated with sterile saline and closed layer by layer using 2-0 Vicryl and 3-0 Nylon.

Fibrin glue (Beriplast P, Germany) was prepared before use by mixing fibrinogen (100 mg/ml) with aprotinin solution (200 KIU/ml) plus calcium chloride (8 mM) and aFGF (1 mg/ml), and applied to the surgical area to form a glue cast. The final glue volume was about 10 μL. The fibrin glue was applied to perineurium. The same surgical procedure was performed for group 2 patients, except that neither fibrin glue nor aFGF was added. Group 3 patients received no surgical intervention.

### Data Analysis

Comparison of Average Muscle Strength Among Three Groups

Comparison of average muscle strength among three groups, both at baseline and follow-up examinations, was made by Kruskal-Wallis test.

Analysis Using GEE Methods with “Average Muscle Strength” as the Outcome Variable

To take into account the repeated measurements' dependency, we used the generalized estimating equations (GEE) method (Diggle et al., 1994, Analysis of Longitudinal Data. Oxford: Clarendon Press; Liang, 1986, Biometria 73: 13-22) to examine the improvements in average muscle strength by testing for changes over time among these 3 groups (changes from baseline evaluation to about 6-month follow-up and to about 12-month follow-up). Moreover, the GEE method's multiple linear regression was able to establish the dependent variables (average muscle strength score) as a function of gender, age, site, sensory function, operation time, axonal continuity and severity graded by the electrophysiological examination.

### Results

Patient Demographics

Table 1 demonstrates patient demographic characteristics of the three groups. A total of 21 patients were included in group 1. There were 14 men and 7 women, ranging in age from 5 to 78 years (mean 37.4 yr). Among them, two patients (9.5%) received nerve graft, neurolysis and fibrin glue mixture (which includes aFGF) after the surgery. Others received neurolysis and fibrin glue mixture (which includes aFGF) after the surgery. In group 2, a total of 8 patients were included, with 5 men and 3 women. The patients' ages ranged from 7 to 81 years (means, 45.6 yr). One patient (12.5%) received neurolysis and nerve graft, while the remaining 7 patients received neurolysis only. Group 3 included 12 men and 4 women. The patients' ages ranged from 13 to 64 years (means, 30.6 yr). No surgical intervention was performed in group 3.

TABLE 1
Patient Characteristics

| | Patient | | Sex | | Injury Etiology | | | | | Type of Operation | | | |
|---|---|---|---|---|---|---|---|---|---|---|---|---|---|
| Group | Number | Mean Age (range) | 0 | 1 | 1 | 2 | 3 | 4 | 5 | 1 | 2 | 3 | 4 |
| 1 | 21 | 37.4 (5 to 78) | 7 | 14 | 9 | 7 | 2 | 1 | 2 | 2 | 19 | 0 | 0 |
| 2 | 8 | 45.6 (7 to 87) | 3 | 5 | 6 | 1 | 0 | 0 | 1 | 0 | 0 | 1 | 7 |
| 3 | 16 | 30.6 (13 to 64) | 4 | 12 | 8 | 7 | 0 | 0 | 1 | 0 | 0 | 0 | 0 |

(Sex: 0 = female, 1 = male; injury etiology: 1 = combined stretch and crush injuries, 2 = stretch or compression injury, 3 = crush injury, 4 = stab injury, 5 = unknown; type of operation: 1 = neurolysis, nerve graft and fibrin glue mixture, 2 = neurolysis and fibrin glue mixture, 3 = neurolysis and nerve graft, 4 = neurolysis)

Comparison of Average Muscle Strength Among Three Groups

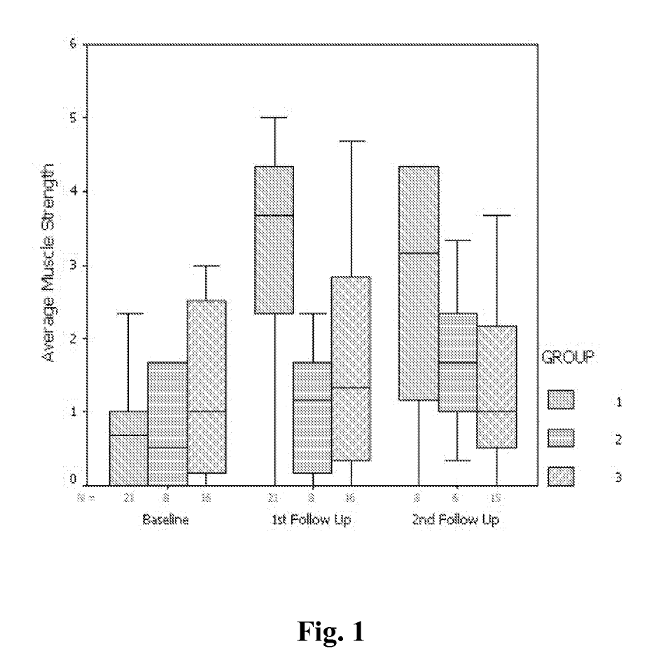

FIG. 1 shows the average muscle strength scores (mean and standard deviation) in each group. The average muscle strength scores by Kruskal-Wallis test (Table 2) were also compared. During baseline evaluation, there was no significant difference of average muscle strength scores among these three groups (p=0.539). The first follow-up evaluation showed that the average muscle strength scores in group 1 (3.06±1.60) was significantly higher than group 2 (1.04±0.86) and group 3 (1.65±1.43) (p=0.005). During the second follow-up evaluation, although group 1 patients had higher average muscle strength score, significant difference was not achieved (p=0.169).

TABLE 2
Average muscle in each three groups and the comparison
of average muscle strength by Kruskal-Wallis test

| | Group 1 | | Group 2 | | Group3 | | P value by Kruskal- |
|---|---|---|---|---|---|---|---|
| | n | mean ± SD | n | mean ± SD | n | mean ± SD | Wallis test |
| Baseline | 21 | 0.98 ± 1.08 | 8 | 0.75 ± 0.83 | 16 | 1.29 ± 1.20 | 0.539 |
| 1^{st} Follow up | 21 | 3.06 ± 1.60 | 8 | 1.04 ± 0.86 | 16 | 1.64 ± 1.43 | 0.005 |
| 2^{nd} Follow up | 8 | 2.71 ± 1.71 | 6 | 1.72 ± 1.04 | 15 | 1.40 ± 1.228 | 0.169 |

Relationship Between Average Muscle Strength and Axonal Continuity as Well as Sensory Function

By GEE method, after adjusting for the effects of age, gender, sensory impairment and the operation of time, the average muscle strength scores of patients with loss of axon continuity was significantly lower than those without loss of axon continuity (p-value=0.008). Likewise, it was also shown that patients with absent sensory function had significant lower average muscle strength scores than those with intact sensory function (p<0.001). Patients with impaired sensory function also had lower average muscle strength scores, but no significance was achieved (p=0.0724).

Comparison of Increase of Average Muscle Strength Score in Each Group

Group 1: Compared to the baseline evaluation, there was significant increase in average muscle strength score by 0.4299 during the first follow-up evaluation and by 0.5045 during the second follow-up evaluation (p=0.0197 and 0.0297, respectively).

Group 2: Compared to the baseline evaluation, average muscle strength score decreased by 0.3141 during the first and by 0.4155 during the second follow-up evaluation, but no significant decrease was attained compared to baseline evaluation (p=0.1380 and p=0.0970, respectively).

Group 3: Compared to the baseline evaluation, average muscle strength score decreased by 0.4997 during the first follow-up evaluation, with borderline significance achieved (p=0.0439); and decreased by 0.4787 during the second follow-up evaluation, while no significant decrease was achieved (p=0.088).

### Results

This study demonstrated significant motor recovery in patients with severe CPN lesions with axon loss after surgical repair using fibrin glue added with aFGF, compared with those after conservative observation or surgical intervention without the treatment of fibrin glue added with aFGF. This is the first human study with respect to the potential of aFGF to facilitate functional recovery of peripheral nerve lesions.

In this study, patients received no surgical intervention (group 3) demonstrated no significant improvement of motor function. Instead, the average muscle strength score decreased by 0.4997 during 1st follow-up evaluation with borderline significance (p=0.0439). During 2nd follow-up evaluation, average muscle strength score decreased by 0.4783 (p=0.088). Our data indicated that patients with severe axon loss showed no significant spontaneous motor recovery if no surgical intervention was performed. Several factors may contribute to the deterioration of motor function in the recovery phase: First, persistent foot drop may cause secondary stretch injury; secondly, scar tissue formation may hinder nerve regeneration and impair the function recovery (Garozzo et al., 2004, supra).

Group 2 patients received conventional surgical intervention without fibrin glue containing aFGF. By GEE method, the average muscle strength score showed no significant difference during the first and second follow-up evaluations compared to the baseline evaluation. This suggested that in severe common peroneal nerve lesions with axon loss, surgical repair only might not lead to significant motor recovery. The large proportion (six out of the eight) of combined stretch and crush injury in Group 2 might also explain the poor surgical outcome.

In group 1 patients, surgical repair coupled with fibrin glue containing aFGF to facilitate nerve growth and motor recovery was used. Compared to the baseline evaluation, there was significant increase in average muscle strength scores during follow-up evaluations. Group 1 patients achieved significant increase in average muscle strength score at 6 months postsurgery compared to the other groups. No significant increase was noted at the 2nd follow-up examination for the group 1 patients. This may be due to the short half life of aFGF. Like other neurotrophic factors, which in general have poor pharmacokinetic profiles and short in vivo half-life, the half-life of aFGF is of only 3.5 days. Previous study using [¹²⁵I]-labeled GDNF suggested that fibrin glue is an effective substrate for keeping a trophic factor localized in situ for a finite period, protected from the circulation, surrounding aqueous humor or CSF (Cheng et al., 1998, Cell Transplant 7: 53-61). Therefore, we included aFGF in the fibrin glue to allow slow release of the growth factor. Continuous supplement of neurotrophic factors might be preferred for continuous regeneration. In addition, this may be due to decreased sample size since a large proportion of patients in group 1 lost follow-up during 2nd follow-up evaluation (lost about 61.9% sample size).

The double-blind controlled model was not feasible in this study since most patients were reluctant to be enrolled randomly. Therefore, our data of patients in groups 2 and 3 were collected retrospectively from previous chart records due to this ethic concern. In this study, there was no significant difference among the baseline average muscle strength scores across three groups. Therefore, selection bias might not be significant despite the fact that our subjects were not recruited blindly and randomly. Our results demonstrated that using a fibrin glue mixture containing a growth factor such as aFGF after a surgical repair (group 1) more effectively repaired CPN lesions and restored the function of the damaged CPN, compared to surgical repair alone (group 2) or without surgical repair (group 3).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method for repairing a common peroneal nerve (CPN) lesion comprising:
i) surgically repairing the CPN at or near the CPN lesion; and
ii) applying an effective amount of a fibrin glue mixture to the surgically repaired area of the CPN, wherein the fibrin glue mixture comprises growth factor, fibrinogen, aprotinin and divalent calcium ions.

2. The method of claim 1, wherein the growth factor is selected from the group consisting of a glial cell line-derived neurotrophic factor, a transforming growth factor-beta, a fibroblast growth factor (FGF), a platelet-derived growth factor, an epidermal growth factor, a vascular endothelial growth factor, a neurotrophin, and combinations thereof.

3. The method of claim 2, wherein the growth factor comprises a fibroblast growth factor (FGF).

4. The method of claim 3, wherein the fibroblast growth factor (FGF) comprises acidic FGF (aFGF).

5. The method of claim 1, wherein the step of surgically repairing comprises a procedure selected from the group consisting of axotomy, nerve graft, neurolysis, and combinations thereof.

6. The method of claim 1, wherein the components of the fibrin glue mixture are applied to the surgically repaired area of the CPN simultaneously or separately.

7. The method of claim 1, wherein the divalent calcium ions are provided by calcium chloride or calcium carbonate.

8. The method of claim 1, wherein the fibrin glue mixture comprises acidic fibroblast growth factor (aFGF), fibrinogen, aprotinin and calcium chloride.

9. The method of claim 8, wherein the fibrin glue mixture comprises, per milliliter volume of the fibrin glue mixture, about 0.0001 to 1000 mg of acidic fibroblast growth factor (aFGF), about 10 to 1000 mg of fibrinogen, about 10 to 500 KIU of aprotinin and about 1 to 100 μmol of calcium chloride.

10. The method of claim 9, wherein the fibrin glue mixture comprises, per milliliter volume of the fibrin glue mixture, about 1 mg of aFGF, about 100 mg of fibrinogen, about 200 KIU of aprotinin and about 8 μmol of calcium chloride.

11. A method for enhancing the functional recovery of a surgically repaired common peroneal nerve (CPN) comprising the step of applying an effective amount of a fibrin glue mixture to the surgically repaired area of the CPN, wherein the fibrin glue mixture comprises growth factor, fibrinogen, aprotinin and divalent calcium ions.

12. The method of claim 11, wherein the growth factor is selected from the group consisting of a glial cell line-derived neurotrophic factor (GDNF), a transforming growth factor-beta, a fibroblast growth factor (FGF), a platelet-derived growth factor, an epidermal growth factor, a vascular endothelial growth factor, a neurotrophin, and combinations thereof.

13. The method of claim 12, wherein the growth factor comprises a fibroblast growth factor (FGF).

14. The method of claim 13, wherein the fibroblast growth factor (FGF) comprises acidic FGF (aFGF).

15. The method of claim 11, wherein the surgically repaired CPN is surgically repaired by a procedure selected from the group consisting of axotomy, nerve graft, neurolysis, and combinations thereof.

16. The method of claim 11, wherein the components of the fibrin glue mixture are applied to the surgically repaired area of the CPN simultaneously or separately.

17. The method of claim 11, wherein the divalent calcium ions are provided by calcium chloride or calcium carbonate.

18. The method of claim 11, wherein the fibrin glue mixture comprises acidic fibroblast growth factor (aFGF), fibrinogen, aprotinin and calcium chloride.

19. The method of claim 18, wherein the fibrin glue mixture comprises, per milliliter volume of the fibrin glue mixture, about 0.0001 to 1000 mg of acidic fibroblast growth factor (aFGF), about 10 to 1000 mg of fibrinogen, about 10 to 500 KIU of aprotinin and about 1 to 100 μmol of calcium chloride.

20. The method of claim 19, wherein the fibrin glue mixture comprises, per milliliter volume of the fibrin glue mixture, about 1 mg of aFGF, about 100 mg of fibrinogen, about 200 KIU of aprotinin and about 8 μmol of calcium chloride.

21. A kit comprising a fibrin glue mixture that comprises growth factor, fibrinogen, aprotinin and divalent calcium ions, and instructions for using the fibrin glue mixture in a surgery repair of a common peroneal nerve lesion.

22. The kit of claim 21, wherein the fibrin glue mixture comprises acidic fibroblast growth factor (aFGF).

23. The kit of claim 21, wherein the fibrin glue mixture comprises, per milliliter volume of the fibrin glue mixture, about 0.0001 to 1000 mg of acidic fibroblast growth factor (aFGF), about 10 to 1000 mg of fibrinogen, about 10 to 500 KIU of aprotinin and about 1 to 100 μmol of calcium chloride.

24. The mixture of claim 23, wherein the fibrin glue mixture comprises, per milliliter volume of the fibrin glue mixture, about 1 mg of aFGF, about 100 mg of fibrinogen, about 200 KIU of aprotinin and about 8 μmol of calcium chloride.
